(19) United States
(12) Patent Application Publication
(10) Pub. No.: US 2015/0290220 A1
(43) Pub. Date: Oct. 15, 2015

(54) Pharmaceutical formualtions comprising a Gluococorticosteroid

(71) Applicants: Arvind Gannimitta, Hyderabad (IN); Sachin Jain, Hyderabad (IN); Sagar Rase, Hyderabad (IN); Jayant Karajgi, Hyderabad (IN); Sivakumaran Meenakshisunderam, Hyderabad (IN)

(72) Inventors: Arvind Gannimitta, Hyderabad (IN); Sachin Jain, Hyderabad (IN); Sagar Rase, Hyderabad (IN); Jayant Karajgi, Hyderabad (IN); Sivakumaran Meenakshisunderam, Hyderabad (IN)

(21) Appl. No.: 14/683,105

(22) Filed: Apr. 9, 2015

(30) Foreign Application Priority Data
Apr. 10, 2014 (IN) IN 1890/CHE/2014

(51) Int. Cl. A61K 31/58

(52) U.S. Cl. CPC A61K 31/58

(57) ABSTRACT

Sterile formulation comprising glucocorticosteroids, process for the preparation of the sterile formulation and method of using the same are provided. The present invention also relates to sterile formulation comprising budesonide, process for the preparation of the sterile formulation and method of using the same.

### CROSS REFERENCE TO RELATED APPLICATIONS

This application claims priority from an Indian Patent Application IN 1890/CHE/2014 filed Apr. 10, 2014.

### FIELD OF THE INVENTION

The present invention relates to a sterile formulation comprising steroids, such as glucocorticosteroids. The present invention also relates to a process for the preparation of such sterile formulations. The present invention also relates to process of sterilization of glucocorticosteroids, in particular in form of suspensions that are intended for use in nebulizers. The present invention also relates to methods of prophylaxis, amelioration and/or treatment of allergic and/or inflammatory conditions of the nose or lungs using sterile formulation of the glucocorticosteroid.

### BACKGROUND OF THE INVENTION

Asthma is a common chronic inflammatory disease of the airways characterized by variable and recurring symptoms, reversible airflow obstruction and bronchospasm. Common symptoms include wheezing, coughing, chest tightness, and shortness of breath. Asthma is thought to be caused by a combination of genetic and environmental factors. Its diagnosis is usually based on the pattern of symptoms, response to therapy over time and spirometry. It is clinically classified according to the frequency of symptoms, forced expiratory volume in one second (FEV1), and peak expiratory flow rate. Asthma may also be classified as atopic (extrinsic) or non-atopic (intrinsic) where atopic refers to a predisposition toward developing type 1 hypersensitivity reactions.

Treatment of acute symptoms is usually with an inhaled short-acting beta-2 agonist (such as salbutamol) and oral corticosteroids. Symptoms can be prevented by avoiding triggers, such as allergens and irritants, and by the use of inhaled corticosteroids. Budesonide is a glucocorticoid steroid for the treatment of asthma, COPD and non-infectious rhinitis (including hay fever and other allergies), and for treatment and prevention of nasal polyposis. In addition, it is used for Crohn's disease (inflammatory bowel disease). Budesonide, the active component of PULMICORT RESPULES®, is a corticosteroid designated chemically as (RS)-11β,16α,17,21-tetrahydroxypregna-1,4-diene-3,20-dione cyclic 16,17-acetal with butyraldehyde. Budesonide is provided as a mixture of two epimers (22R and 22S). The empirical formula of budesonide is C25H34O6 and its molecular weight is 430.5. Its structural formula is:

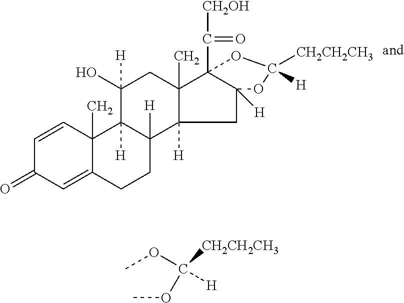

Pulmicort® Respules is a sterile suspension for inhalation via jet nebulizer and contains the active ingredient budesonide (micronized), and the inactive ingredients disodium edetate, sodium chloride, sodium citrate, citric acid, polysorbate 80, and Water for Injection. Three dose strengths are available in single-dose ampules (Respules™ ampules): 0.25 mg, 0.5 mg, and 1 mg per 2 mL RESPULES ampule. For Pulmicort® Respules, like all other nebulized treatments, the amount delivered to the lungs will depend on patient factors, the jet nebulizer utilized, and compressor performance. Using the Pari-LC-Jet Plus Nebulizer/Pari Master compressor system, under in vitro conditions, the mean delivered dose at the mouthpiece (% nominal dose) was approximately 17% at a mean flow rate of 5.5 L/min. The mean nebulization time was 5 minutes or less. Pulmicort® Respules should be administered from jet nebulizers at adequate flow rates, via face masks or mouthpieces.

Inhalation preparations are very much prone to microbial attack and may undergo degradation if not prepared under strict guidelines prescribed by the FDA. Previously it was acceptable for drugs intended for use in nebulizers to be prepared under “clean” conditions. However U.S. FDA has implemented a requirement for all nebulizer solutions to be sterile. In view of this U.S. FDA requirement it is necessary to produce sterile suspension drugs in the U.S.

Sterilization is a process performed to ensure that there is complete freedom from microbial contamination. Sterilization is especially done for pharmaceutical formulations which are to be directly introduced into the body and its cavities. Such formulations explicitly include ophthalmic preparations, nasal preparations, ocular preparations, injectables and the like. Such sterilized preparations involve two main methods of preparation. First route is that the active ingredient is sterilized and the formulation is prepared aseptically or the final is prepared, packed in the desired container and then sterilized. The second route is known as a terminal sterilization technique. This technique refers to the complete destruction of all living microorganisms.

Sterilization of materials relies on the input of sufficient energy to be lethal to any potential microbial contamination. Numerous methods including heat, radiation, and chemicals have been proposed for the sterilization of glucocorticosteroids. However, to date these methods often result in the excess production of degradants or a loss of activity for the glucocorticosteroid being sterilized. Additionally, as in the case of glucocorticosteroid suspension formulations for metered dose inhalation, the commonly used sterilization procedures often results in unacceptable changes to drug particle size.

Several methods have been proposed in the past for the sterilization of glucocorticosteroids. PT-A-69652 discloses the cold sterilization of micronized glucocorticosteroids using mixtures of ethylene oxide and carbon dioxide. However, ethylene oxide is toxic and when it is used to sterilize glucocorticosteroids it has been found that the residual amounts of the ethylene oxide contravene pharmaceutical guidelines which require very low levels of residual ethylene oxide. Accordingly this method has been found to be unsuitable for producing therapeutically acceptable glucocorticosteroids and formulations thereof.

U.S. Pat. No. 3,962,430 discloses a method for the production of sterile isotonic solutions of medicinal agents. The method comprises adding the medicinal agent to a saturated solution of sodium chloride in water at 100° C. and then heating the mixture to 100-130° C. This method is not suitable for suspensions of fine particles of glucocorticosteroids intended for inhalation, as the procedure produces unfavorable changes in the size of the particles. Indeed this method can lead to the formation of bridges between the fine particles producing large, hard aggregates, which do not break into the desired fine particles up on administration.

A putative alternative is dry heat sterilization. According to the European Pharmacopoeia (1996, pp. 283-4) a normal heat sterilization process runs at 180° C. for 30 min or at a minimum of 160° C. for at least 2 hours. According to Pharmacopoeia Nordica (1964, pp. 16) such a sterilization can be carried out at 140° C. for 3 hours. However at the temperatures of these processes glucocorticosteroids suffer significant degradation and are subject to changes in their surface structure.

Sterilization by β or γ-irradiation is also known. Indeed Illum and Moeller in Arch. Pharm. Chemi. Sci., Ed. 2, 1974, pp. 167-174 recommend the use of such irradiation to sterilize glucocorticosteroids. However when such irradiation is used to sterilize certain finely divided, e.g. micronized, glucocorticosteroids, they are significantly degraded.

U.S. Pat. No. 6,392,036 discloses a process for the sterilization of a powder comprising a glucocorticosteroid which comprises heat treating the powder at a temperature of from 100° C. to 130° C. This patent further discloses that the glucocorticosteroid is preferably used in the form of a finely divided, e.g. micronized, powder, particularly in the form of finely divided particles having a mass median diameter of less than 10 μm, more preferably less than 5 μm.

U.S. Pat. No. 6,863,865 discloses a method of sterilizing a pharmaceutical composition, comprising rapidly heating the pharmaceutical composition from ambient temperature to an elevated temperature, maintaining it at or above the elevated temperature for a period of time, and rapidly cooling the pharmaceutical composition to an ambient temperature, wherein the pharmaceutical composition is or contains a suspension of a glucocorticosteroid. This patent further discloses that the particle size of the budesonide was such that 100% of the particles were less than 10 μm and 95% of the particles were less than 5 μm in diameter.

U.S. Pat. No. 7,892,483 discloses a process for the sterilization of a steroid which process comprises heat treating the steroid in the form of a wet mass consisting essentially of the steroid, water and surfactant, wherein the water is not saturated with respect to any solute present in the water, wherein the amount of water in the wet mass is no more than ten times the amount of steroid and wherein at the end of sterilization the sterilized mass comprises at least 1%, by weight of the total mass, of water. This patent further discloses that the steroid is preferably in finely divided particulate form, with 90% of the particles preferably having a diameter of less than 10 μm. More preferably, 90% of the particles have a diameter of less than 5 μm.

U.S. Pat. No. 8,178,519 discloses a method for preparing a sterile suspension of a glucocorticosteroid comprising (i) heating a glucocorticosteroid suspension comprising a glucocorticosteroid, water and a surfactant in a mixing vessel to sterilize the glucocorticosteroid suspension, re-circulating the glucocorticosteroid suspension via a homogenizer before, during and/or after step (ii), and subsequently (iii) mixing the glucocorticosteroid suspension with sterile water or a sterile excipient liquid comprising water and one or more pharmaceutically acceptable excipients; wherein the heating is carried out for at least 2 minutes.

US 2008/0139519 discloses a method for the sterilization of a labile glucocorticosteroid, comprising the step of applying moist heat to a suspension of a labile glucocorticosteroid for a sterilizing-effective time, wherein at least 70% of the glucocorticosteroid is in the form of a suspension during heating and at least one surfactant is present in the aqueous suspension during heating.

The above prior arts employ sterilized glucocorticosteroid which is free of pyrogens and bacterial endotoxins for the manufacturing of formulation and thus special precaution must be taken during handling of active and other inactive ingredients to minimize microbial contamination. Further, aseptic processing is required throughout the manufacturing operation wherein all vessels and equipments used for the manufacturing of the formulation must be sterilised, movement of personnel must be controlled and methodical, and special attention must be paid to the validation and monitoring of the entire manufacturing process which in turn leads to high processing cost and eventually may place the sterility of formulation in jeopardy.

However, the inventors of the present invention have endeavored to develop alternate practicable process wherein budesonide is heat sterilised and/or milled during the manufacturing of formulation and not initially. This, in turn, helps to lower processing costs and provides a higher sterility assurance level. Further, a narrow particle size distribution of budesonide is obtained which demonstrates comparable stability and bioavailability to the marketed formulation of Pulmicort® Respules.

### OBJECTIVE OF THE INVENTION

Accordingly, an objective of the present invention is to provide pharmaceutical formulations containing glucocorticosteroid and one or more pharmaceutically acceptable excipients.

Another objective of the present invention is to provide sterile formulation containing glucocorticosteroid and one or more pharmaceutically acceptable excipients.

Yet another objective of the present invention is to provide process for preparation of sterile pharmaceutical formulation containing glucocorticosteroid.

Yet another objective of the present invention is to provide methods for using sterile formulation containing glucocorticosteroid.

### SUMMARY OF THE INVENTION

An aspect of the present invention is to provide a pharmaceutical formulation comprising glucocorticosteroid, surfactant, water and optionally one or more pharmaceutically acceptable excipients.

In another aspect of the present invention relates to a pharmaceutical formulation comprising budesonide, at least one surfactant, water and optionally one or more pharmaceutically acceptable excipients.

In another aspect, the present invention relates to a pharmaceutical formulation comprising unmicronised budesonide, at least one surfactant, water and optionally one or more pharmaceutically acceptable excipients.

In another aspect, the present invention relates to a pharmaceutical formulation comprising micronised budesonide, at least one surfactant, water and optionally one or more pharmaceutically acceptable excipients.

In another aspect, the present invention relates to a pharmaceutical formulation which is prepared by initially taking non-sterile budesonide as the input active agent, at least one surfactant, water and optionally one or more pharmaceutically acceptable excipients, and wherein the said formulation is sterilized only after its preparation.

In another aspect, the present invention relates to a pharmaceutical formulation which is prepared by initially taking non-sterile unmicronised budesonide as the input active agent, at least one surfactant, water and optionally one or more pharmaceutically acceptable excipients, and wherein the said formulation is sterilized only after its preparation.

In another aspect, the present invention relates to a pharmaceutical formulation which is prepared by initially taking non-sterile micronised budesonide as the input active agent, at least one surfactant, water and optionally one or more pharmaceutically acceptable excipients, and wherein the said formulation is sterilized only after its preparation.

In another aspect, the present invention relates to a pharmaceutical formulation which is prepared by initially taking sterile budesonide as the input active agent, at least one surfactant, water and optionally one or more pharmaceutically acceptable excipients, and wherein the said formulation is optionally sterilized after its preparation.

In another aspect, the present invention relates to a pharmaceutical formulation which is prepared by initially taking sterile unmicronised budesonide as the input active agent, at least one surfactant, water and optionally one or more pharmaceutically acceptable excipients, and wherein the said formulation is optionally sterilized after its preparation.

In another embodiment, the present invention relates to a process for the sterilization of the input active agent budesonide and/or the formulation after its preparation, wherein said process comprises a method other than the heat treatment method.

In another embodiment, the present invention relates to a process for the sterilization of budesonide formulation which comprises heat treating the homogenized slurry comprising budesonide, at least one surfactant and water.

In another aspect, the present invention relates to a process for the sterilization of budesonide formulation wherein the process comprises heat treating the formulation followed by homogenizing the formulation aseptically.

In another aspect, the present invention relates to a process for the sterilization of budesonide formulation, wherein the process comprises homogenizing the formulation followed by heat treating the formulation.

In another aspect, the present invention relates to a process for the preparation of a sterile formulation wherein the process comprises of the following steps:
(i) dispersing budesonide in a solution comprising water and optionally one or more pharmaceutically acceptable excipients to form a slurry,
(ii) optionally sterilizing and/or homogenizing the slurry of step (i), and
(iii) mixing the slurry of step (ii) with sterile excipient liquid comprising water and optionally one or more pharmaceutically acceptable excipients to obtain the sterile formulation.

In another aspect, the present invention relates to a process for the preparation of a sterile formulation wherein the process comprises of the following steps:
(i) dispersing budesonide in a solution comprising water and optionally one or more pharmaceutically acceptable excipients to form a slurry,
(ii) sterilizing the slurry of step (i),
(iii) homogenizing the slurry of step (ii) aseptically, and
(iv) mixing the slurry of step (iii) with sterile excipient liquid comprising water and optionally one or more pharmaceutically acceptable excipients to obtain the sterile formulation.

In another aspect, the present invention relates to a process for the preparation of a sterile formulation wherein the process comprises of the following steps:
(i) dispersing budesonide in a solution comprising water and optionally one or more pharmaceutically acceptable excipients to form a slurry,
(ii) homogenizing the slurry of step (i),
(iii) sterilizing the slurry of step (ii), and
(iv) mixing the slurry of step (iii) with sterile excipient liquid comprising water and optionally one or more pharmaceutically acceptable excipients to obtain the sterile formulation.

An aspect of the present invention relates to methods of prophylaxis, amelioration and/or treatment of allergic and/or inflammatory conditions by administering to a subject in need thereof a sterile formulation comprising budesonide, at least one surfactant, water and optionally one or more pharmaceutically acceptable excipients. Another aspect of the present invention relates to method of prophylaxis, amelioration and/or treatment of asthma by administering to a subject in need thereof a sterile formulation comprising budesonide, at least one surfactant, water and optionally one or more pharmaceutically acceptable excipients.

### DETAILED DESCRIPTION OF THE INVENTION

An embodiment of the present invention is to provide a pharmaceutical formulation comprising glucocorticosteroid, surfactant, water and one or more pharmaceutically acceptable excipients.

In another embodiment, the present invention relates to a pharmaceutical formulation comprising sterile budesonide, at least one surfactant, water and one or more pharmaceutically acceptable excipients.

In another embodiment, the present invention relates to a pharmaceutical formulation comprising unmicronised budesonide, at least one surfactant, water and one or more pharmaceutically acceptable excipients.

In another embodiment, the present invention relates to a pharmaceutical formulation comprising micronised budesonide, at least one surfactant, water and one or more pharmaceutically acceptable excipients.

In another aspect, the present invention relates to a pharmaceutical formulation which is prepared by initially taking non-sterile micronized or unmicronised budesonide as the input active agent, at least one surfactant, water and optionally one or more pharmaceutically acceptable excipients, and wherein the said formulation is sterilized only after its preparation.

In another aspect, the present invention relates to a pharmaceutical formulation which is prepared by initially taking sterile unmicronised budesonide as the input active agent, at least one surfactant, water and optionally one or more pharmaceutically acceptable excipients, and wherein the said formulation is optionally sterilized after its preparation.

In another embodiment, the present invention relates to a process for the sterilization of the input active agent budesonide and/or the formulation after its preparation, wherein said process comprises a method other than the heat treatment method.

In another embodiment, the present invention relates to a process for the sterilization of budesonide formulation which comprises heat treating the homogenized slurry comprising budesonide, at least one surfactant and water.

In another aspect, the present invention relates to a process for the sterilization of budesonide formulation wherein the process comprises heat treating the formulation followed by homogenizing the formulation aseptically.

In another embodiment, the present invention relates to a process for the sterilization of budesonide formulation, wherein the process comprises homogenizing the formulation followed by heat treating the formulation.

In another embodiment, the present invention relates to a process for the preparation of a sterile formulation wherein the process comprises of the following steps:
(i) dispersing budesonide in a solution comprising water and optionally one or more pharmaceutically acceptable excipients to form a slurry,
(ii) optionally sterilizing and/or homogenizing the slurry of step (i), and
(iii) mixing the slurry of step (ii) with sterile excipient liquid comprising water and optionally one or more pharmaceutically acceptable excipients to obtain the sterile formulation.

In another embodiment, the present invention relates to a process for the preparation of a sterile formulation wherein the process comprises of the following steps:
(i) dispersing budesonide in a solution comprising water and optionally one or more pharmaceutically acceptable excipients to form a slurry,
(ii) sterilizing the slurry of step (i),
(iii) homogenizing the slurry of step (ii) aseptically, and
(iv) mixing the slurry of step (iii) with sterile excipient liquid comprising water and optionally one or more pharmaceutically acceptable excipients to obtain the sterile formulation.

In another embodiment, the present invention relates to a process for the preparation of a sterile formulation wherein the process comprises of the following steps:
(i) dispersing budesonide in a solution comprising water and optionally one or more pharmaceutically acceptable excipients to form a slurry,
(ii) homogenizing the slurry of step (i),
(iii) sterilizing the slurry of step (ii), and
(iv) mixing the slurry of step (iii) with sterile excipient liquid comprising water and optionally one or more pharmaceutically acceptable excipients to obtain the sterile formulation.

In the specification and the appended claims, singular forms, including the singular forms “a,” “an” and “the”, specifically also encompass the plural referents of the terms to which they refer unless the context clearly dictates otherwise. In addition, as used herein, unless specifically indicated otherwise, the word “or” is used in the “inclusive” sense of “and/or” and not the “exclusive” sense of “either/or.”

As used in this specification, whether in a transitional phrase or in the body of a claim, the terms “comprise(s)” and “comprising” are to be interpreted as having an open-ended meaning That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

The term “sterile” means that a product or composition meets the criteria of sterility according to the US Pharmacopoeia 27/NF22, 2004, or its counterpart in other jurisdictions, and which provides a therapeutically acceptable glucocorticosteroid and/or pharmaceutical formulation.

As used herein, “glucocorticosteroid” refers to any of a group of steroid hormones (including derivatives, synthetic analogs, and pro-drugs), such as cortisone, which are produced by the adrenal cortex. Non-limiting examples of glucocorticosteroids, which may be used in the present invention, include beclomethasone, budesonide, ciclesonide, cortivazol, deflazacort, flumethasone, flunisolide, fluocinolone, fluticasone, mometasone, rofleponide, tipredane and triamcinolone. Preferably, use is made of budesonide, beclomethasone (e.g. the dipropionate), ciclesonide, fluticasone, mometasone and triamcinolone.

To obtain an efficient dispersion of the glucocorticosteroid particles in the solution, a surfactant is used. The surfactants may also function as stabilizing agents in the formulations according to the present invention. Examples of suitable surfactants are selected from a group comprising but not limited to tyloxapol a polymer of 4-(1,1,3,3-tetramethylbutyl)phenol with ethylene oxide, formaldehyde sorbitan derivatives, e.g. polyoxyethylene sorbitan esters, preferably of the polysorbate groups, more preferably polysorbate 80 or polysorbate 20, polyoxyethylene ethers, especially polyoxyethylene alkyl ethers, poloxamers, polyoxyethylene castor oil derivatives, polyvinylalcohol and block copolymers of polyethyleneoxides, polypropyleneoxides, polybutyleneoxides, polyethyleneglycols (PEGs), polyethylene glycol derivatives, especially polyethylene glycol 660 hydroxystearate or Soluto™ HS 15 and mixtures thereof. The surfactant may be present at about 0.002 to 2% w/w of the formulation.

The pharmaceutically acceptable excipients according to present invention is selected from a group comprising but not limited to a group consisting of surfactants, pH regulating agents, chelating agents and agents rendering the suspension isotonic, and mixtures thereof.

Suitable pH regulating agent according to present invention is selected from but not limited to the group comprising weak organic acids such as citric acid anhydrous, citric acid monohydrate and the like, strong mineral acids such as hydrochloric acid and the like, and strong alkaline agents such as NaOH and the like thereof. Alternatively, the pH of the system can be adjusted by balancing the acid and salt forms of buffers such as citric acid, sodium citrate, acetic acid, sodium acetate and sodium phosphate. The pH of the formulation is in the range of from about 3.5 to about 6.0, more preferably from 4.0 to 5.0, and most preferably from 4.2 to 4.8.

Suitable chelating agent according to present invention includes disodium edetate (EDTA) and the like. The chelating agent may be present at about 0.005 to 0.1% w/w of the formulation.

Suitable isotonic agent according to present invention is selected from the group comprising dextrose, glycerol, mannitol, sodium chloride, potassium chloride and sodium bromide and the like.

In an embodiment budesonide is present in the slurry at a concentration of about 0.1 mg/ml to about 15 mg/ml. In further embodiment budesonide is present at a concentration of about 0.5 mg/ml to about 10 mg/ml.

The glucocorticosteroid according to the present invention has the volume mean size ‘D(90)’ of about 5 to 250 μm, or about 15 to 100 μm, or about 30 to 60 μm. Furthermore, the ‘D(100)’ value of the volume mean particle size distribution is from about 10 to 300 μm, or about 30 to 200 μm, or about 60 to 100. The unmicronised glucocorticosteroid according to the present invention has the volume mean size ‘D(90)’ of about 25 to 250 μm, or about 30 to 100 μm, or about 30 to 60 μm. Furthermore, the ‘D(100)’ value of the volume mean particle size distribution is from about 40 to 300 μm, or about 50 to 200 μm, or about 60 to 100. The particle size can be measured by any of the commonly known methods such as by light scattering techniques, for example, by Malvern or Horiba equipments. The slurry comprising unmicronised glucocorticosteroid, surfactant and water is then milled/homogenized in a homogenizer to reduce the particle size distribution of glucocorticosteroid to a prespecified value.

According to an embodiment of the present invention, the glucocorticosteroid after homogenization is preferably in a finely divided particulate form, with 90% of the particles having a diameter of less than about 20 μm, or less than about 10 μm.

The homogenizer is a device known in the art in which a suspension or slurry of a particulate material, here the glucocorticosteroid suspension, is subjected to an energetic shear as the suspension is forced to pass therethrough. The homogenizer provides a sufficiently high shear force to cause the breakup of aggregates of particles in the suspension and a reduction in the solid particles sizes. A precise numerical range for the level of shear is not appropriate given that the level of shear will depend on the viscosity of the suspension. The homogenizer 10 may be an in-line high-shear homogenizer (e.g. a Silverson 150 L) or, for more efficient and better particle size reduction, a high-pressure homogenizer (e.g. a Niro Panda). A high-shear homogenizer typically has a mixing workhead comprising rotatable rotor blades and a perforated stator with the rotor blades located within the stator. A high-pressure homogenizer typically comprises a pump, which can supply pressures up to about 1500 bar, and one or more interaction chambers where the passage of fluid through minute flow passages under high pressure and controlled flow action subjects the fluid to conditions of high turbulence and shear.

The present invention relates to a method of sterilizing or heat treating glucocorticosteroids that are susceptible to higher temperatures. The method involves introducing the glucocorticosteroid into a pressure vessel or other sealed container along with one or more excipients (preferably surfactant) and water. The pressure vessel is preferably fitted with a hydrophobic vent filter and a hydrophobic cartridge filter. The sterilization or heat treating is preferably done at temperatures ranging from about 70° C. to about 150° C. or 101° C. to about 145° C. for about 2 minutes to about 180 minutes at varying pressures. For example, a combination of temperature-time-pressure includes 121° C. for 30 minutes at 15 psi. Generally, the higher the temperature and pressure, the shorter the time required for adequate sterilization.

The sterilization can also be done by the other methods including, but not limited to heat sterilization, chemical sterilization, gaseous sterilization, radiation sterilization & sterile filtration.

In an embodiment, the sterilization is done by heat treatment, such as by using dry heat or autoclaving.

In another embodiment, the sterilized budesonide slurry is then mixed with sterile excipient liquid comprising water and optionally one or more pharmaceutically acceptable excipients.

In another embodiment, the excipient liquid may be sterilized either by filtration via a sterilizing grade filter or by autoclaving.

In another embodiment, the sterile excipient liquid is transferred aseptically to the sterile glucocorticoid slurry and then mixed with sterile water for injection.

In an embodiment the sterile formulation is in the form of suspension.

In another embodiment, the suspension of the present invention contains from about 0.05 to about 20 mg/ml of the glucocorticosteroid, or from about 0.08 to 10 mg/ml of the glucocorticosteroid, or from about 0.1 to 5 mg/ml of the glucocorticosteroid.

The sterile suspension of present invention is then filled into suitable container using BFS (Blow Fill Seal) machine. The BFS machine may use any pharmaceutically acceptable primary container material.

Typically low-density polyethylene granulate is used to form the primary container/closure system on the BFS machine although high-density polyethylene, polypropylene, poly vinyl chloride or polyethylene terephthalate may also be used. Mixtures of these materials may also be used.

The BFS machine is configured to present open topped units to the filling head for each machine filling cycle. The sterile glucocorticosteroid suspension is filled into the formed units through a time/pressure/dosing unit which delivers a precise measure of the suspension via filling needles. Following filling the filling needles are withdrawn and the head section of the mould closes to seal the units completely. The filled units are then removed from the BFS machine.

In an embodiment the present invention relates to methods of prophylaxis, amelioration and/or treatment of allergic and/or inflammatory conditions by administering to a subject in need thereof a sterile formulation comprising budesonide, at least one surfactant, water and optionally one or more pharmaceutically acceptable excipients. In another embodiment the present invention relates to method of prophylaxis, amelioration and/or treatment of asthma by administering to a subject in need thereof a sterile formulation comprising budesonide, at least one surfactant, water and optionally one or more pharmaceutically acceptable excipients.

The following examples further exemplify the invention and are not intended to limit the scope of the invention in any manner whatsoever. It is obvious to those skilled in the art to find out the composition for other dosage forms and substitute the equivalent excipients as described in this specification or with the one known to the industry.

### Examples 1-3

| | | Example-1 | Example-2 | Example-3 |
|---|---|---|---|---|
| S. No. | Ingredients | Mg/ml | Mg/ml | Mg/ml |
| 1 | Budesonide | 0.125 | 0.25 | 0.50 |
| 2 | Edetate disodium | 0.10 | 0.10 | 0.10 |
| 3 | Sodium Chloride | 8.50 | 8.50 | 8.50 |
| 4 | Sodium Citrate | 0.50 | 0.50 | 0.50 |
| 5 | Citric Acid Anhydrous | 0.28 | 0.28 | 0.28 |
| 6 | Polysorbate 80 | 0.20 | 0.20 | 0.20 |
| 7 | Water for injection (WFI) | q.s to 1 mL | q.s to 1 mL | q.s to 1 mL |

The processing steps involved in manufacturing sterile suspension of budesonide as given in examples 1 to 3 are given below:
(i) Required quantity of Water for Injection was collected in to a vessel based on the batch size.
(ii) The collected Water for Injection of step (i) was cooled to 20° C. to 30° C. and then sparged with Nitrogen till the dissolved oxygen content was less than about 2 ppm.
(iii) Water for Injection approximately 10% of total batch size was collected in a vessel. To this, Edetate disodium and Polysorbate 80 were added and dissolved.
(iv) To the solution of step (iii), Budesonide was added and dispersed.
(v) The slurry of step (iv) was then homogenized using high pressure homogenizer.
(vi) The homogenized slurry of step (v) was then autoclaved at 121° C. for 30 minutes and then cooled to room temperature under stirring.
(vii) Water for Injection in a quantity of approximately 70% of total batch size was collected in a vessel.
(viii) Dispensed quantity of sodium chloride, sodium citrate and citric acid were added one after another in to the Water for Injection of step (vii), under continuous stirring to get clear solution.
(ix) The solution of step (viii) was filtered and added aseptically to the sterilized slurry of step (vi) under continuous stirring to get uniform suspension.
(x) The volume was made up to 100% of batch size with the remaining filtered Water for Injection under continuous stirring to get uniform suspension.

### Examples 4-6

| | | Example-4 | Example-5 | Example-6 |
|---|---|---|---|---|
| S. No. | Ingredients | Mg/ml | Mg/ml | Mg/ml |
| 1 | Budesonide | 0.125 | 0.25 | 0.50 |
| 2 | Edetate disodium | 0.10 | 0.10 | 0.10 |
| 3 | Sodium Chloride | 8.50 | 8.50 | 8.50 |
| 4 | Sodium Citrate | 0.50 | 0.50 | 0.50 |
| 5 | Citric Acid Anhydrous | 0.28 | 0.28 | 0.28 |
| 6 | Polysorbate 80 | 0.20 | 0.20 | 0.20 |
| 7 | Water for injection (WFI) | q.s to 1 mL | q.s to 1 mL | q.s to 1 mL |

The processing steps involved in manufacturing sterile suspension of budesonide as given in examples 4 to 6 are given below:
(i) Required quantity of Water for Injection was collected in to a vessel based on the batch size.
(ii) The collected Water for Injection of step (i) was cooled to 20° C. to 30° C. and then sparged with Nitrogen till the dissolved oxygen content was less than about 2 ppm.
(iii) Water for Injection approximately 10% of total batch size was collected in a vessel. To this, Edetate disodium and Polysorbate 80 were added and dissolved.
(iv) To the solution of step (iii), Budesonide was added and dispersed.
(v) The slurry of step (iv) was then autoclaved at 121° C. for 30 minutes and then cooled to room temperature under stirring.
(vi) The slurry of step (v) was then homogenized using high pressure homogenizer under aseptic conditions.
(vii) Water for Injection in a quantity of approximately 70% of total batch size was collected in a vessel.
(viii) Dispensed quantity of sodium chloride, sodium citrate and citric acid were added one after another in to the Water for Injection of step (vii), under continuous stirring to get clear solution.
(ix) The solution of step (viii) was filtered and added aseptically to the sterilized slurry of step (vi) under continuous stirring to get uniform suspension.
(x) The volume was made up to 100% of batch size with the remaining filtered Water for Injection under continuous stirring to get uniform suspension.

### Examples 7-9

| | | Example-7 | Example-8 | Example-9 |
|---|---|---|---|---|
| S. No. | Ingredients | Mg/ml | Mg/ml | Mg/ml |
| 1 | Budesonide | 0.125 | 0.25 | 0.50 |
| 2 | Edetate disodium | 0.10 | 0.10 | 0.10 |
| 3 | Sodium Chloride | 8.50 | 8.50 | 8.50 |
| 4 | Sodium Citrate | 0.50 | 0.50 | 0.50 |
| 5 | Citric Acid Monohydrate | 0.28 | 0.28 | 0.28 |
| 6 | Polysorbate 80 | 0.20 | 0.20 | 0.20 |
| 7 | Water for injection (WFI) | q.s to 1 mL | q.s to 1 mL | q.s to 1 mL |

The processing steps involved in manufacturing the sterile suspension of budesonide as given in examples 7 to 9 are given below:
(i) Required quantity of Water for Injection was collected in to a vessel based on the batch size.
(ii) The collected Water for Injection of step (i) was cooled to 20° C. to 30° C. and then sparged with Nitrogen till the dissolved oxygen content was less than 1.5 ppm.
(iii) Water for Injection approximately 40% of total batch size was collected in a vessel. To this, Edetate disodium, Polysorbate 80, sodium chloride, sodium citrate and citric acid was added and dissolved under continuous stirring to get clear solution.
(iv) The solution of step (iii) was filtered.
(v) To the solution of step (iv), Budesonide was added and dispersed.
(vi) The volume was made up to 100% of batch size with the remaining part of filtered Water for Injection under continuous stirring to get uniform suspension.
(vii) The suspension of step (vi) was bulk sterilized at 121° C. for 30 minutes by passing steam in jacket of the vessel.
(viii) The suspension was then cooled to room temperature.
(ix) The suspension of step (viii) was then homogenized in homogenizer.

We claim:

1. A process for the preparation of a sterile formulation wherein the process comprises of the following steps:
(i) dispersing budesonide in a solution comprising water and optionally one or more pharmaceutically acceptable excipients to form a slurry,
(ii) optionally sterilizing and/or homogenizing the slurry of step (i), and
(iii) mixing the slurry of step (ii) with sterile excipient liquid comprising water and optionally one or more pharmaceutically acceptable excipients to obtain the sterile formulation.

2. The process according to claim 1, wherein the process comprises of the following steps:
(i) dispersing budesonide in a solution comprising water and optionally one or more pharmaceutically acceptable excipients to form a slurry,
(ii) sterilizing the slurry of step (i),
(iii) homogenizing the slurry of step (ii) aseptically, and
(iv) mixing the slurry of step (iii) with sterile excipient liquid comprising water and optionally one or more pharmaceutically acceptable excipients to obtain the sterile formulation.

3. The process according to claim 1, wherein the process comprises of the following steps:
(i) dispersing budesonide in a solution comprising water and optionally one or more pharmaceutically acceptable excipients to form a slurry,
(ii) homogenizing the slurry of step (i),
(iii) sterilizing the slurry of step (ii), and
(iv) mixing the slurry of step (iii) with sterile excipient liquid comprising water and optionally one or more pharmaceutically acceptable excipients to obtain the sterile formulation.

4. The process according to claim 1, wherein the budesonide is micronised and non-sterile.

5. The process according to claim 1, wherein the budesonide is unmicronised and sterile.

6. The process according to claim 1, wherein the budesonide is unmicronised and non-sterile.

7. The process according to claim 1, wherein the pharmaceutically acceptable excipient is selected from a group comprising surfactants, pH regulating agents, chelating agents and agents rendering the suspension isotonic, and mixtures thereof.

8. The process according to claim 7, wherein the surfactant is selected from a group comprising tyloxapol, polyoxyethylene sorbitan esters, polyoxyethylene ethers, poloxamers, polyoxyethylene castor oil derivatives, polyvinylalcohol and block copolymers of polyethyleneoxides, polypropyleneoxides, polybutyleneoxides, polyethyleneglycols (PEGs), polyethylene glycol derivatives, and mixtures thereof.

9. The process according to claim 1, wherein the budesonide is dispersed in a solution comprising surfactant, water and one or more pharmaceutically acceptable excipients to form the slurry.

10. The process according to claim 2, wherein the slurry is sterilized by heat treatment.

11. The process according to claim 3, wherein the slurry is sterilized by heat treatment.

12. The process according to claim 10, wherein the heat treatment is carried out at a temperature of from about 70° C. to about 150° C. for about 2 minutes to about 180 minutes.

13. The process according to claim 11, wherein the heat treatment is carried out at a temperature of from about 70° C. to about 150° C. for about 2 minutes to about 180 minutes.

14. The process according to claim 1, wherein the budesonide has a D(90) particle size of about 5 μm to about 250 μm.

15. The process according to claim 1, wherein the budesonide has a D(90) particle size of about 25 μm to about 250 μm.

16. The process according to claim 1, wherein the budesonide is present in the slurry at a concentration of about 0.1 mg/ml to about 15 mg/ml.

17. A method of using the sterile formulation prepared by the process according to claim 1 for prophylaxis, amelioration, treatment of allergic or inflammatory conditions, or for treatment of asthma.

18. A method of treating allergic or inflammatory conditions or treating asthma, by administering the sterile formulation prepared by the process according to claim 1 to a subject in need thereof.
